(19) United States
(12) Patent Application Publication
(10) Pub. No.: US 2019/0062362 A1
(43) Pub. Date: Feb. 28, 2019

(54) METHOD FOR OBTAINING SURFACTANT COMPOSITIONS MADE FROM ALKYL-L-GULURONAMIDES

(71) Applicant: ÉCOLE NATIONALE SUPÉRIEURE DE CHIMIE, Rennes (FR)

(72) Inventors: Thierry BENVEGNU, Rennes (FR); Nouha SARI-CHMAYSSEM, Rennes (FR); Samir TAHA, Beirut (LB); Hiba MAWLAWI, Tripoli (LB)

(73) Assignee: ÉCOLE NATIONALE SUPÉRIEURE DE CHIMIE, Rennes (FR)

(21) Appl. No.: 16/061,176

(22) PCT Filed: Dec. 9, 2016
(86) PCT No.: PCT/FR2016/053290

§ 371 Date: Nov. 14, 2018

(30) Foreign Application Priority Data
Dec. 11, 2015 (FR) 1562228

(51) Int. Cl. C07H 15/04; B01F 17/00; C11D 1/825; C11D 1/66; C07H 1/00; A01N 43/16; A01N 43/08

(52) U.S. Cl. CPC C07H 15/04; B01F 17/0092; B01F 17/0042; A01N 43/08; C11D 1/662; C07H 1/00; A01N 43/16; C11D 1/825

(57) ABSTRACT

Some embodiments relate to a novel method for obtaining surfactant compositions made from alkyl-L-guluronamides or mixtures of L-guluronamides and D-mannuronamides, the compositions obtained by the method and the uses thereof.

### CROSS REFERENCE TO RELATED APPLICATIONS

This application is a national phase filing under 35 C.F.R. § 371 of and claims priority to PCT Patent Application No. PCT/FR2016/053290, filed on Dec. 9, 2016, which claims the priority benefit under 35 U.S.C. § 119 of French Patent Application No. 1562228, filed on Dec. 11, 2015, the contents of each of which are hereby incorporated in their entireties by reference.

### BACKGROUND

Some embodiments relate to a novel direct process for obtaining surfactant compositions including alkyl L-guluronamides or mixtures of alkyl L-guluronamides and D-mannuronamides from biosourced starting materials (alginates, oligoalginates, poly(oligo)guluronates, brown algae) or biocompatible/biodegradable starting materials.

Some embodiments find applications, for example, in surfactants, especially for cosmetology and the plant protection, agrifood and detergency (industrial) fields.

In the description below, the references in parentheses ([ ]) refer to the list of references presented at the end of the text.

Carbohydrate-based surfactants represent an important class of amphiphilic compounds whose growing interest may be explained by functional, economic and environmental factors (Hill and Lehen-Ferrenbach, 2009) [1]. Sugar amide derivatives characterized by the presence of an amide function connecting the hydrophilic sugar head to the lipophilic chain have the advantage of being resistant to hydrolysis in neutral and alkaline media, especially when compared with ester derivatives (Laurent et al., 2011) [2]. Although studies have already shown the possibility of gaining access to amide derivatives from uronic acids such as glucuronic acid and galacturonic acid derived from the hydrolysis of hemicelluloses or pectins (Laurent et al., 2011, cited above) [2], few studies enabling the viable exploitation of polysaccharides of algal origin exist. Only one example of an amide surfactant is derived from the transformation of D-mannuronic acid oligomers originating from the depolymerization of alginates. On the other hand, the preparation of surfactant compositions in amide form based on L-guluronic acid or mixtures of L-guluronic acid and of D-mannuronic acid which can enable all of the saccharides present in the biopolymer to be viably exploited has not been developed to date.

Three distinct classes of saccharide-based surfactants exist: esters (sorbitan esters, sucroesters), acetals (alkylpolyglucosides) and amides (alkyl glucamides). Industrially, alkyl sucroamides are produced in two steps: reductive amination of a carbohydrate with an alkylamine, followed by acylation of the resulting N-glycoside (international patent application WO 92/06984; international patent application WO 93/03004; U.S. Pat. No. 7,655,611; U.S. Pat. No. 5,872,111) [3-6]. Similarly, gluconamides are obtained in two steps: oxidation of a carbohydrate leading to a lactone or an aldonic acid followed by reaction with alkylamines to form gluconamides (U.S. Pat. No. 2,670,345) [7]. Derivatives including an amide bond between the hydrophilic and lipophilic parts via an N-glycoside bond have more recently been developed (U.S. Pat. No. 7,655,611 cited above) [5]. Another strategy is based on the formation of N-alkylamide surfactants from uronic acids such as glucuronic acid and galacturonic acid derived from the hydrolysis of hemicelluloses or pectins (Laurent et al., 2011, cited above) [2]. All these surfactant synthesis processes use monosaccharides as starting materials and the synthetic conditions are generally sparingly environmentally friendly (toxic and non-biodegradable reagents).

Mannuronamide surfactants have been produced from D-mannuronic acid oligomers (Benvegnu and Sassi, Topics in Current Chemistry, 294: 143-164, 2010; international patent application WO 03/104 248) [8, 9]. The process is based on the production of saturated oligomannuronates (acidic depolymerization), which are then transformed into a monosaccharide intermediate including two butyl chains. This synthon is then subjected to an aminolysis reaction using a fatty amine in a solvent such as methanol or isopropanol in the presence or absence of an organic base. The N-acyl derivative thus obtained has emulsifying properties. However, the use of poly(oligo)mers based on L-guluronic acid originating from the depolymerization of alginates (international patent application WO 03/099 870) [10] or from the whole alginate has not been viably exploited to date.

### SUMMARY

Some embodiments are directed to a novel process for synthesizing compounds and compositions which address or overcome the defects, drawbacks and obstacles of the related art, in particular for a process for controlling the industrial-scale production, reducing the costs and improving the expected properties of compounds and compositions especially in the field of surfactants, and which satisfy the principle of “blue chemistry”.

The applicants have developed a novel solvent-free process, using biocompatible/biodegradable reagents, for affording access simply to surfactant compositions based on L-guluronamide or mixtures of L-guluronamides and of D-mannuronamides directly (“one-pot” process) from poly(oligo)guluronates, bacterial alginates, refined and semi-refined alginates (mixture of alginate, cellulose, hemicellulose and fucan) or brown algae, thus avoiding a preliminary depolymerization step. The poly(oligo)guluronates originate from the depolymerization of alginates, for example according to the process described in international patent application WO 03/099 870 [10]. The alginates (semi-refined, refined) and the oligoalginates are obtained via simple treatments in acidic aqueous media, from fresh or dried algae, obtained, for example, according to the protocol described in example 2 below according to the process of international patent application WO 98/40511 [12]. The bacterial alginates are obtained, for example, from mucoid bacterial cultures (e.g. cf. international patent application WO 2009/134 368) [11].

Some embodiments are directed to a process for obtaining a composition including:

(i) alkyl L-guluronamides of formulae (Ia) and (Ib):

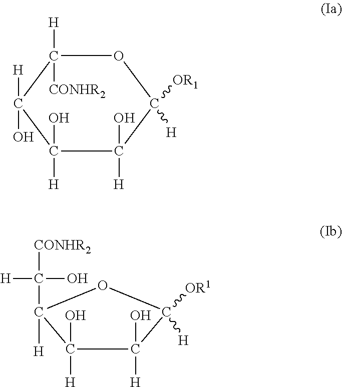

(ii) a mixture of alkyl L-guluronamides of formulae (Ia) and (Ib) and of alkyl D-mannuronamides of formulae (IIa) and (IIb):

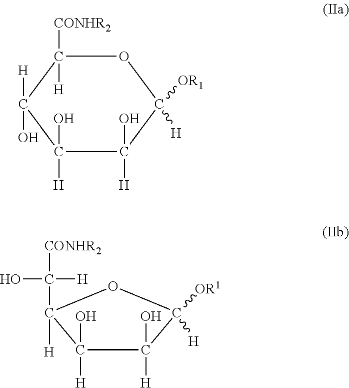

in which:
R₁ is a linear or branched, saturated or unsaturated alkyl chain of 2 to 22, advantageously or preferably 2 to 8 and preferentially 2 to 4 carbon atoms;
R₂ is a hydrogen atom, a linear or branched, saturated or unsaturated alkyl chain of 2 to 22, advantageously or preferably 8 to 18 and preferentially 12 to 18 carbon atoms which may include a terminal amine function; the method including:
   a) a butanolysis and Fischer glycosylation reaction step using poly(oligo)guluronates, alginates, oligoalginates, and/or brown algae; and
   b) aminolysis reaction on the reaction medium derived from step a), in the presence of an amine of formula R′NH₂ in which R′ is composed of 2 to 22, advantageously or preferably 8 to 18 and preferentially 12 to 18 linear or branched, saturated or unsaturated carbon atoms.

For the purposes of some embodiments, the term “poly(oligo)guluronates” means homopolymeric blocks of α-L-guluronic acid partly in sodium salt form derived from the depolymerization of alginates, for example according to the process of international patent application WO 03/099 870 [10].

For the purposes of some embodiments, the term “oligoalginates” means products derived from an enzymatic and/or acidic treatment of alginate, obtained, for example, according to the protocol described in example 2 below according to the process of international patent application WO 98/40511 [12].

For the purposes of some embodiments, the term “alginates” are intended to refer to refined and/or semi-refined alginates, obtained, for example, according to the protocol described in example 2 below. Bacterial alginates are also intended, obtained, for example, from mucoid bacterial cultures (e.g. cf. international patent application WO 2009/134 368) [11]. For the purposes of some embodiments, the term “brown algae” means the algae named Phaeophyceae or Pheophyceae, of which 1500 species exist (e.g. Ascophyllum nodosum, Fucus serratus, Laminaria hyperborea, Laminaria digitate, Ecklonia maxima, Macrocystis pyrifera, Sargassum vulgare, etc. . . . ), and the walls of which are essentially composed of fucan sulfates and alginate.

According to a particular embodiment of some embodiments, the process includes, before step a), the steps of preparing the (semi-)refined alginates, oligoalginates and poly(oligo)guluronates. The poly(oligo)guluronates originate from the depolymerization of alginates. The semi-refined alginates originate from the acidic leaching of brown algae followed by dissolution of the sodium alginates by increasing the pH followed by solid/liquid separation so as to remove the algal residues. The refined alginates originate from an additional depigmentation step with formaldehyde and a purification step. The oligoalginates originate from the enzymatic and/or acidic treatment of alginate solution. The bacterial alginates are obtained, for example, from mucoid bacterial cultures (e.g. cf. international patent application WO 2009/134 368) [11].

According to a particular embodiment of some embodiments, the process may also include a step a′) of neutralizing the reaction medium derived from step a), and performed before step b), leading to a final composition including a variable amount of residual fatty amine salt. For example, the neutralization step is performed in the presence of 1M sodium hydroxide, up to a pH of 7.

According to a particular embodiment of some embodiments, the butanolysis and Fischer glycosylation step a) is performed in the presence (i) of water and/or an ionic solvent and/or a eutectic solvent, (ii) of a linear or branched, saturated or unsaturated alcohol ROH, containing from 1 to 4 carbon atoms, advantageously or preferably n-butanol, and (iii) of an acid catalyst, for instance hydrochloric acid, sulfuric acid, an alkylsulfuric acid such as decyl or lauryl sulfuric acid, a sulfonic acid such as benzenesulfonic acid, para-toluenesulfonic acid, camphorsulfonic acid, an alkylsulfonic acid such as methylsulfonic acid, decylsulfonic acid, laurylsulfonic acid, sulfosuccinic acid or an alkyl sulfosuccinate such as decyl sulfosuccinate or lauryl sulfosuccinate, perhalohydric acids, such as perchloric acid, of metals such as iron, oxides thereof or salts thereof, such as the halides thereof. Advantageously or preferably, it is an alkylsulfonic acid or methanesulfonic acid.

For the purposes of some embodiments, the term “ionic solvent” means, for example, 1-butyl-3-methylimidazolium chloride [BMIM]Cl, 1-butyl-3-methylimidazolium bromide [BMIM]Br, tris(2-hydroxyethyl)-methylammonium methyl sulfate (HEMA) or 1-ethyl-3-methylimidazolium acetate [EMIM]AcO; the ionic solvent typically including up to 10% of water.

For the purposes of some embodiments, the term “eutectic solvent” means systems formed from a eutectic mixture of Lewis or Brønsted bases or acids which may contain a variety of anionic species and/or of cationic species. The first-generation eutectic solvents were based on mixtures of quaternary ammonium salts with hydrogen bonding donors such as amines and carboxylic acids (e.g. quaternary ammonium salt and metal chloride (hydrate).

This step a) is performed, for example, by placing in contact one equivalent of poly(oligo)guluronates with a degree of polymerization of between 2 and 100, advantageously or preferably about 35, derived from the acidic depolymerization (international patent application WO 03/099 870) [10] of alginates extracted from the species Ascophyllum, Durvillaea, Ecklonia, Laminaria, Lessonia, Macrocystis, Sargassum and Turbinaria, and advantageously or preferably of alginates rich in L-guluronic acid; from 10 to 1000 molar equivalents of water; from 2 to 300 molar equivalents of alcohol, such as n-butanol, are introduced, and advantageously or preferably 150 molar equivalents; from 10⁻³ to 10 molar equivalents of an acid catalyst, such as hydrochloric acid, sulfuric acid, an alkylsulfuric acid such as decylsulfuric or laurylsulfuric acid, a sulfonic acid such as benzenesulfonic acid, para-toluenesulfonic acid, camphorsulfonic acid, an alkylsulfonic acid such as methylsulfonic acid, decylsulfonic acid, laurylsulfonic acid, sulfosuccinic acid or an alkyl sulfosuccinate such as decyl sulfosuccinate or lauryl sulfosuccinate, perhalohydric acids, such as perchloric acid, of metals such as iron, oxides thereof or salts thereof, such as the halides thereof, and advantageously or preferably from 1.1 to 10 molar equivalents of alkylsulfonic acid, and advantageously or preferably 2.1 molar equivalents of methylsulfonic acid.

The reaction is then performed at the reflux point of the azeotrope at atmospheric pressure (Dean-Stark apparatus), between 130 and 135° C. in the case of butanol, advantageously or preferably over 12 hours. The composition thus formed is predominantly constituted by compounds bearing two chains originating from the alcohol (advantageously or preferably butanol) derived from L-guluronic acid.

The preparation of the alkyl L-guluronamides in which the alkyl chain is derived from a fatty amine proceeds via the aminolysis step b), after lowering the temperature (advantageously or preferably to 60° C.), by adding from 1 to 25 molar equivalents of a linear or branched, saturated or unsaturated amine of formula R′NH₂, in which R′ is composed of 5 to 22 carbon atoms, and advantageously or preferably 3 molar equivalents are added.

The reaction is performed at a temperature advantageously or preferably of 65-70° C. and under reduced pressure for the recycling of the alcohol mentioned previously.

The composition thus formed constitutes a customary product derived from L-guluronic acid such as emulsifiers.

The unreacted salts and sugars may be removed from this composition by taking up in an organic solvent, advantageously or preferably diethyl ether, and then filtered off and rinsed several times with the organic solvent. The filtrate containing the alkyl L-guluronamides is concentrated to give a composition enriched in products of interest which also constitutes a customary product such as an emulsifier with antibacterial and antifungal properties at the concentrations used for the formation of the emulsions.

Step a) of the process of some embodiments is performed, for example, by placing in contact one equivalent of alginate, such as the alginates extracted from the species Ascophyllum, Durvillaea, Ecklonia, Laminaria, Lessonia, Macrocystis, Sargassum and Turbinaria, oligoalginates derived from a depolymerization, and advantageously or preferably alginates rich in L-guluronic acid; from 10 to 1000 molar equivalents of water; from 2 to 300 molar equivalents of alcohol, such as n-butanol, are introduced, and advantageously or preferably 150 molar equivalents; from 10⁻³ to 10 molar equivalents of an acid catalyst, such as hydrochloric acid, sulfuric acid, an alkylsulfuric acid such as decylsulfuric or laurylsulfuric acid, a sulfonic acid such as benzenesulfonic acid, para-toluenesulfonic acid, camphorsulfonic acid, an alkylsulfonic acid such as methylsulfonic (or methanesulfonic) acid, decylsulfonic acid, laurylsulfonic acid, sulfosuccinic acid or an alkyl sulfosuccinate such as decyl sulfosuccinate or lauryl sulfosuccinate, perhalohydric acids, such as perchloric acid, of metals such as iron, oxides thereof or salts thereof, such as the halides thereof, and advantageously or preferably from 1.1 to 10 molar equivalents of alkylsulfonic acid, and advantageously or preferably 2.5 molar equivalents of methylsulfonic acid.

The reaction is then performed at the reflux point of the azeotrope at atmospheric pressure (Dean-Stark apparatus), between 130 and 135° C. in the case of butanol, advantageously or preferably over 24 hours.

The composition thus formed is predominantly constituted by compounds bearing two chains originating from the alcohol (advantageously or preferably butanol) derived from L-guluronic acid and D-mannuronic acid.

The preparation of the alkyl L-guluronamides and of the alkyl D-mannuronamides in which the alkyl chain is derived from a fatty amine proceeds via the aminolysis step b), after lowering the temperature (advantageously or preferably to 60° C.), according to two possible protocols:

1) The aminolysis reaction is performed without prior neutralization of the medium: in the presence of 1 to 25 molar equivalents of a linear or branched, saturated or unsaturated amine of formula R′NH₂, in which R′ is composed of 2 to 22 carbon atoms, and advantageously or preferably of 3 molar equivalents. For example, the fatty amine is chosen from the group constituted by dodecylamine and oleylamine. The reaction is performed at a temperature advantageously or preferably of 65-70° C. and under reduced pressure for the recycling of the alcohol mentioned previously. The composition thus formed constitutes a customary product derived from L-guluronic acid and D-mannuronic acid such as emulsifiers. The unreacted salts and sugars may be removed from this composition by taking up in an organic solvent, advantageously or preferably diethyl ether, and then filtered off and rinsed several times with the organic solvent. The filtrate containing the alkyl L-guluronamides and the alkyl D-mannuronamides is concentrated to give a composition enriched in products of interest which also constitutes a customary product such as an emulsifier with antibacterial and antifungal properties at the concentrations used for the formation of the emulsions.

2) The aminolysis reaction is performed after preliminary neutralization of the medium: by adding 1N NaOH solution to a pH close to 7. The medium is then concentrated six-fold under reduced pressure without reducing it to dryness. Next, 1 to 10 molar equivalents of a linear or branched, saturated or unsaturated amine of formula R′NH₂, in which R′ is composed of 5 to 22 carbon atoms, and advantageously or preferably of 1 molar equivalent, are added. For example, the fatty amine is chosen from the group constituted by dodecylamine and oleylamine. The reaction is performed at a temperature advantageously or preferably of 65-70° C. and under reduced pressure for the recycling of the alcohol mentioned previously. Next, from 100 to 1000 molar equivalents of water, advantageously or preferably 500 equivalents, are added to the medium. The mixture is stirred for about 15 minutes at 65-70° C. After stopping the stirring, the medium is left for about 10 minutes at this temperature so that the organic products flocculate. After lowering the temperature to room temperature, the organic phase solidifies and it is then easy to remove the water containing salts.

The compositions thus formed via the process of some embodiments constitute customary products derived from L-guluronic acid and D-mannuronic acid such as emulsifiers with antibacterial and antifungal properties at the concentrations used for the formation of the emulsions.

Some embodiments are also directed to a composition obtained via the process according to some embodiments. The compositions of some embodiments are constituted by L-guluronic acid derivatives or of two uronic acid derivatives (L-guluronic acid and D-mannuronic acid) derived from the same polysaccharide. In addition, the L-guluronic acid and D-mannuronic acid derivatives which constitute the compositions of some embodiments are simultaneously in the form of pyranosides (6-membered ring) and furanosides (5-membered rings). Depending on the chain length and on the nature of the alkyl chains, the compositions of some embodiments will be considered as emulsifiers for water-in-oil (W/O) or oil-in-water (O/W) emulsions. Furthermore, they may have antibacterial and antifungal properties.

Some embodiments are also directed to the use of a composition according to some embodiments as a surfactant. Advantageously or preferably, the surfactant is chosen from the group constituted by solubilizers, hydrotropes, wetting agents, foaming agents, emulsifying agents, emulsifiers and/or detergents.

Some embodiments are also directed to a composition according to some embodiments for use as an antibacterial and/or antifungal agent.

Some embodiments are also directed to a surfactant including a composition according to some embodiments. The surfactant may have the following properties:

| Number of carbon | |
|---|---|
| atoms in the lipophilic | Surfactant bearing two lipophilic chains: alkyl D- |
| chain (alkyl R2): | mannuronamides and/or alkyl L-guluronamides |
| Between 1 and 6 | Hydrotropes and/or solubilizers |
| Between 6 and 14 | Oil-in-water (O/W) and/or water-in-oil (W/O) |
| | emulsifiers |
| Between 16 and 22 | Water-in-oil (W/O) emulsifiers |

Some embodiments are also directed to an antifungal and/or antibacterial including a composition according to some embodiments.

The process of some embodiments leads to novel surfactant compositions by exclusively using biosourced starting materials (poly(oligo)guluronates, alginates, oligoalginates, brown algae) or biocompatible/biodegradable starting materials:
using a methodology which makes it possible to transform the L-guluronic acid originating from poly(oligo)guluronates or simultaneously L-guluronic acid and D-mannuronic acid, i.e. the two constituent uronic sugars of alginates, to give surfactant compositions constituted exclusively of L-guluronic acid or of the two saccharides (L-guluronic acid and D-mannuronic acid);
using conditions which may make it possible to dispense with the preliminary depolymerization of the alginate and thus to use it directly;
proposing conditions which satisfy the principles of blue chemistry, reactions without organic solvents other than the reactive alcohols/amines, not producing any waste (recycling of the short-chain alcohols (n-butanol, etc.)) and using biodegradable reagents (methanesulfonic acid and the like);
performing all the reactions via a “one-pot” process, without isolation or purification of the reaction intermediates;
by controlling the amount of fatty amine used during the aminolysis step and the conditions for neutralization of the acid used during the first phase of the process, it is possible to produce surfactant compositions including variable proportions of alkylammonium salts;
using simple conditions for the partial purification of the crude reaction products and for isolation of the surfactant compositions, which make it possible to produce derivatives and compositions at prices that are more competitive than the current market.

The process of some embodiments makes it possible to produce compositions based on L-guluronic acid or based simultaneously on L-guluronic acid and D-mannuronic acid in amide form, which have the advantage of forming water-in-oil (W/O) and oil-in-water (O/W) emulsions that are very stable in comparison with commercial emulsifiers, and of having antibacterial and antifungal properties at the concentrations used for the formation of the emulsions.

Thus, the process of some embodiments makes it possible simultaneously to reduce the production costs of surfactant compositions and to propose novel compositions for the purpose of improving the performance qualities (especially the emulsifying properties).

Other advantages may also appear to a person of ordinary skill in the art on reading the examples below, which are illustrated by the attached figures, given for illustrative purposes.

### BRIEF DESCRIPTION OF THE FIGURES

FIG. 1 represents the measurement of the interface tensions of compositions GN12, GN18, AlgN12, AlgN18 and crude Algn12.

FIG. 2 represents the measurement of the emulsifier power of compositions GN12, GN18, AlgN12, AlgN18 and crude Algn12 in comparison with the commercial references Xylance® and Montanov®.

### DETAILED DESCRIPTION OF EXEMPLARY EMBODIMENTS

### Examples

### Example 1: Process for Obtaining Alkyl L-Guluronamides from Poly(Oligo)Guluronates

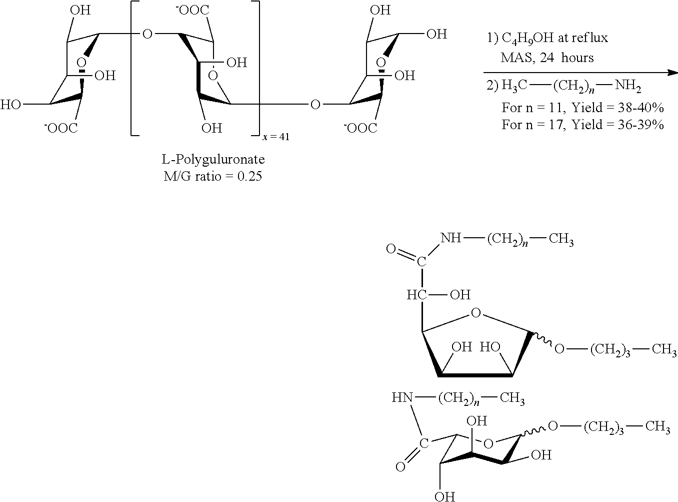

Preparation of the starting materials: the poly(oligo)guluronates may be obtained, for example, according to the process of international patent application WO 03/099 870 [10].

### 1) Butanolysis and Fischer Glycosylation Reaction

1 g of sodium L-poly(oligo)guluronate (7500 g/mol, degree of polymerization=44) whose M/G ratio is 0.25 (5.71 mmol, 1 eq) was mixed with 2 mL of distilled water. 80 mL (150 eq) of butanol were added to the alginate solution with stirring with 778 μl of methanesulfonic acid (12 mmol, 2.1 eq). The medium was stirred at the reflux temperature of butanol (130-135° C.) for 12 hours. The waters present in the medium and those formed during the reaction were removed in Dean-Stark apparatus filled with butanol, via water-butanol azeotropic distillation. Given that it is denser than butanol, the water moves to the bottom of the Dean-Stark apparatus and a few ml of butanol pass into the flask to maintain the initial volume. After 12 hours, thin-layer chromatography (95/5 v/v CH₂Cl₂/CH₃OH) was performed on the reaction medium to ensure that the expected product had indeed been synthesized.

### 2) Aminolysis Reaction

The temperature of the medium was lowered to 60° C., followed by addition of 3 molar equivalents (17.14 mmol) of fatty amine (for n=11, 3.18 g and for n=17, 4.62 g) that may be required to increase the pH to 8.5. After stirring for 30 minutes at 65° C. and under a reduced pressure of 150 mbar, the butanol was evaporated off while reducing the pressure from 150 mbar to 6 mbar over a period of 1 hour. The medium was left under a reduced pressure of 6 mbar for 1 hour 30 minutes to ensure the evaporation of the traces of butanol that were formed.

The residue obtained was taken up in diethyl ether and then filtered through a sinter funnel and washed several times with diethyl ether to remove the salts and the unreacted starting sugar. The filtrate (containing our alkyl guluronamides) was concentrated under vacuum to remove the diethyl ether. A brown oil was thus obtained.

For n=11, after optional chromatography of the oil obtained on a column of silica gel (80 g, using as eluent 95/5 v/v CH₂Cl₂/CH₃OH), the presence of 0.95 g (2.27 mmol, 40% yield) of a mixture of four isomeric forms of N-(12-dodecyl)-n-butyl β-L-gulurofuranosiduronamide (37%), N-(12-dodecyl)-n-butyl α-L-gulurofuranosiduronamide (21%), N-(12-dodecyl)-n-butyl α-L-guluropyranosiduronamide (28%) and N-(12-dodecyl)-n-butyl β-L-guluropyranosiduronamide (14%) was determined. The pyranose/furanose ratio is 0.74. This surfactant composition was named GN12.

For n=17, after optional chromatography of the oil obtained on a column of silica gel (80 g, using as eluent 95/5 v/v CH₂Cl₂/CH₃OH), the presence of 1.08 g (2.16 mmol, 38% yield) of a mixture of four isomeric forms of N-(18-octadecyl)-n-butyl β-L-gulurofuranosiduronamide (36%), N-(18-octadecyI)-n-butyl α-L-gulurofuranosiduronamide (23%), N-(18-octadecyl)-n-butyl α-L-guluropyranosiduronamide (25%) and N-(18-octadecyl)-n-butyl β-L-guluropyranosiduronamide (16%) was determined. The pyranose/furanose ratio is 0.69. This surfactant composition was named GN18.

### Example 2: Process for Obtaining Alkyl L-Guluronamides and D-Mannuronamides from Alginates

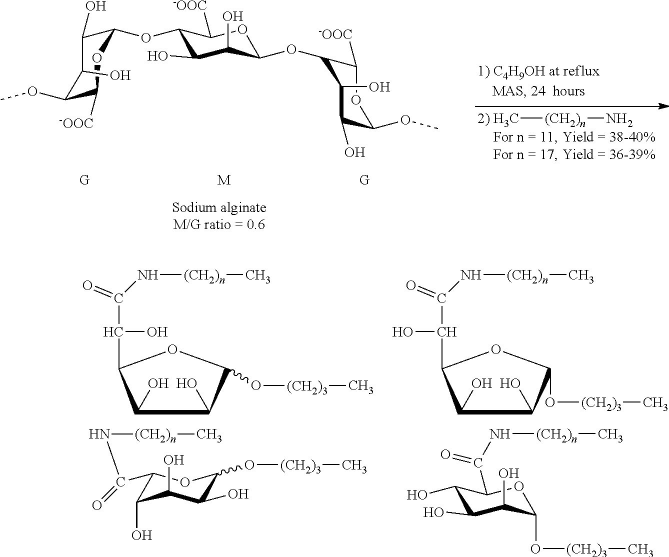

Preparation of the starting materials: Alginate extraction processes are conventionally used at the CEVA (René Perez “La culture des algues marines dans le monde [Cultivation of marine algae throughout the world]”, Ifremer). They involve acidic leaching of fresh or dried algae (washing of the harvested algae with seawater, depigmentation in formaldehyde, grinding, extraction with 0.2N sulfuric acid at room temperature, draining and rinsing of the leached algae with distilled water), followed by dissolution of the sodium alginates by increasing the pH of the medium followed by solid/liquid separation so as to remove the algal residues (addition of a 1.5% Na₂CO₃ solution containing 50 g dry weight of leached algal material at a dry alga/1.5% Na₂CO₃ solution ratio of 0.025, stirring with an IKA reactor for 3 hours at 55° C., cooling in an ice-water bath to avoid excessive temperature differences, centrifugation for 5 minutes at 6000 rpm, and solid/liquid separation). At this stage, the liquid fraction may be frozen and freeze-dried and constitutes the semi-refined alginates in sodium alginate form. In order to obtain refined alginates, purification is performed in the preceding steps. After separation of the algal residues, this final purification step includes or consists of precipitation of the alginic acid by lowering the pH, followed by washing several times with acidic water so as to remove the co-products. Increasing the pH with Na₂CO₃ makes it possible once again to dissolve the sodium alginates while limiting the salts, relative to the use of sodium hydroxide. Lastly, a final step of freezing and then freeze-drying makes it possible to obtain the final product. In order to obtain saturated or unsaturated oligoalginates the alginate solution is treated enzymatically or with acid so as to lower the degree of polymerization of the alginates from 20 to 3.

### A) Without Prior Neutralization of the Reaction Medium Before the Aminolysis Reaction

1) Butanolysis and Fischer Glycosylation Reaction

1 g of sodium alginate (110 200 g/mol, degree of polymerization=630, extracted from Sargassum vulgare) whose M/G ratio is 0.71 (5.71 mmol, 1 eq) was mixed with 3 mL of distilled water. 80 mL (150 eq) of butanol were added to the alginate solution with stirring with 927 μl of methanesulfonic acid (14.27 mmol, 2.5 eq). The medium was stirred at the reflux temperature of butanol (130-135° C.) for 24 hours. The waters present in the medium and those formed during the reaction were removed in Dean-Stark apparatus filled with butanol, via water-butanol azeotropic distillation. Given that it is denser than butanol, the water moves to the bottom of the Dean-Stark apparatus and a few ml of butanol pass into the flask to maintain the initial volume. After 24 hours, thin-layer chromatography (95/5 v/v CH₂Cl₂/CH₃OH) was performed on the reaction medium to ensure that the expected product had indeed been synthesized.

2) Aminolysis Reaction

The temperature of the medium was lowered to 60° C., followed by addition of 3 molar equivalents (17.14 mmol) of fatty amine (for n=11, 3.18 g and for n=17, 4.62 g) that may be required to increase the pH to 8.5. After stirring for 30 minutes at 65° C. and under a reduced pressure of 150 mbar, the butanol was evaporated off while reducing the pressure from 150 mbar to 6 mbar over a period of 1 hour. The medium was left under a reduced pressure of 6 mbar for 1 hour 30 minutes to ensure the evaporation of the traces of butanol that were formed.

The residue obtained was taken up in diethyl ether and then filtered through a sinter funnel and washed several times with diethyl ether to remove the salts and the unreacted starting sugar. The filtrate (containing the alkyl guluronamides and mannuronamides) was concentrated under vacuum to remove the diethyl ether. A dark brown oil is thus obtained.

For n=11, after optional chromatography of the oil obtained on a column of silica gel (80 g, using as eluent 95/5 v/v CH₂Cl₂/CH₃OH), the presence of 0.91 g (2.18 mmol, 39% yield) of a mixture of six isomeric forms (C₂₂H₄₃NO₆, molar mass=417.59 g/mol) of N-(12-dodecyl)-n-butyl β-L-gulurofuranosiduronamide (26%), N-(12-dodecyl)-n-butyl α-L-gulurofuranosiduronamide (15%), N-(12-dodecyl)-n-butyl α-L-guluropyranosiduronamide (16%), N-(12-dodecyl)-n-butyl β-L-guluropyranosiduronamide (12%), N-(12-dodecyl)-n-butyl α-D-mannofuranosiduronamide (18%) and N-(12-dodecyl)-n-butyl α-D-mannopyranosiduronamide (14%) was determined. This surfactant composition was named AlgN12.

After 1D and 2D NMR spectral analysis of the crude reaction product (before purification), the following were calculated: the pyranose/furanose ratio of the guluronamide forms (0.72), the pyranose/furanose ratio of the mannuronamide forms (0.81) and the mannuronamide/guluronamide ratio (0.47).

In addition, the amount of dodecylammonium mesylate salts formed during the aminolysis reaction was able to be quantified. Starting with 1 g of alginate (1 eq), this amount formed was 1.8 molar equivalents (0.010 mol, 2.89 g).

Characterization of the associated amine salt: dodecylammonium mesylate

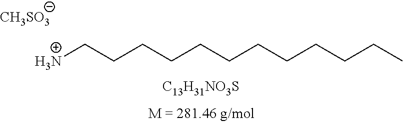

For n=17, after optional chromatography of the oil obtained on a column of silica gel (80 g, using as eluent 95/5 v/v CH₂Cl₂/CH₃OH), the presence of 1.03 g (2.05 mmol, 36% yield) of a mixture of six isomeric forms (C₂₈H₅₅NO₆, molar mass=501.75 g/mol) of N-(18-octadecyl)-n-butyl β-L-gulurofuranosiduronamide (25%), N-(18-octadecyI)-n-butyl α-L-gulurofuranosiduronamide (15%), N-(18-octadecyI)-n-butyl α-L-guluropyranosiduronamide (16%), N-(18-octadecyI)-n-butyl β-L-guluropyranosiduronamide (12%), N-(18-octadecyl)-n-butyl α-D-mannofuranosiduronamide (18%) and N-(18-octadecyI)-n-butyl α-D-mannopyranosiduronamide (14%) was determined. This surfactant composition was named AlgN18.

After 1D and 2D NMR spectral analysis of the crude reaction product (before purification), the following were calculated: the pyranose/furanose ratio of the guluronamide forms (0.71), the pyranose/furanose ratio of the mannuronamide forms (0.84) and the mannuronamide/guluronamide ratio (0.46). Similarly, the presence of the octadecylammonium mesylate salts formed during the aminolysis reaction was able to be quantified. Starting with 1 g of alginate (1 eq), this amount formed was 2 molar equivalents (0.011 mol, 4.16 g).

Characterization of the associated amine salt: octadecylammonium mesylate.

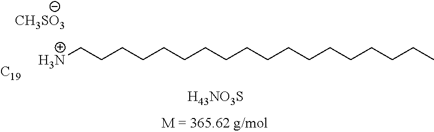

### B) With Prior Neutralization of the Reaction Medium Before the Aminolysis Reaction

For the purpose of developing a process not followed by a purification step, which is easier to perform on a pilot scale, while using a low amount of amine and avoiding the use of organic solvents (except for butanol), the protocol described below was performed:

1) Butanolysis and Fischer Glycosylation Reaction

1 g of sodium alginate (110 200 g/mol, degree of polymerization=630, extracted from Sargassum vulgare) whose M/G ratio is 0.71 (5.71 mmol, 1 eq) was mixed with 3 mL of distilled water. 80 mL (150 eq) of butanol were added to the alginate solution with stirring with 927 μl of methanesulfonic acid (14.27 mmol, 2.5 eq). The medium was stirred at the reflux temperature of butanol (130-135° C.) for 24 hours. The waters present in the medium and those formed during the reaction were removed in Dean-Stark apparatus filled with butanol, via water-butanol azeotropic distillation. Given that it is denser than butanol, the water moves to the bottom of the Dean-Stark apparatus and a few ml of butanol pass into the flask to maintain the initial volume. After 24 hours, thin-layer chromatography (95/5 v/v CH₂Cl₂/CH₃OH) was performed on the reaction medium to ensure that the expected product had indeed been synthesized.

2) Neutralization of the Reaction Medium

In order to reduce the amount of amines to be added during the second step of the process, the reaction medium was neutralized, after cooling, with 1M sodium hydroxide (8 mL) to a pH of 7. Next, the water was evaporated off on a rotary evaporator.

3) Aminolysis Reaction

1 molar equivalent of dodecylamine (5.71 mmol, 1.06 g) was added to the reaction medium. After stirring for 30 minutes at 65° C. and under a reduced pressure of 150 mbar, the butanol was evaporated off while reducing the pressure from 150 mbar to 6 mbar over a period of 1 hour. The medium was left under a reduced pressure of 6 mbar for 1 hour 30 minutes to ensure the evaporation of the traces of butanol that were formed.

A step to remove the salts included or consisted of adding 500 molar equivalents of water (60 mL) and the mixture was stirred at 70° C. for 15 minutes. After stopping the stirring, the amide organic products flocculated at the surface of the water. On leaving the medium to cool to room temperature, the organic phase solidified and it was then easy to remove the water containing salts, and the solid flocculates were recovered. These flocculates, which are an extremely intense brown color, are formed from the crude product of alkyl L-guluronamide and D-mannuronamide and of the dodecylammonium mesylate salts. The final mass of crude product obtained was 1.21 g (2.9 mmol). This surfactant composition was named crude AlgN12.

The presence of a mixture of six isomeric forms (C₂₂H₄₃NO₆, molar mass=417.59 g/mol) of N-(12-dodecyl)-n-butyl β-L-gulurofuranosiduronamide (24%), N-(12-dodecyl)-n-butyl α-L-gulurofuranosiduronamide (14%), N-(12-dodecyl)-n-butyl α-L-guluropyranosiduronamide (18%), N-(12-dodecyl)-n-butyl β-L-guluropyranosiduronamide (10%), N-(12-dodecyl)-n-butyl α-D-mannofuranosiduronamide (19%) and N-(12-dodecyl)-n-butyl α-D-mannopyranosiduronamide (15%) was determined from the analysis of the 1D and 2D NMR spectra of the crude reaction product (without purification).

These data made it possible to calculate the pyranose/furanose ratio of the guluronamide forms (0.73), the pyranose/furanose ratio of the mannuronamide forms (0.79) and also the mannuronamide/guluronamide ratio (0.51).

Furthermore, the presence of the dodecylammonium mesylate salts formed during the aminolysis reaction was able to be quantified. Starting with 1 g of alginate (1 eq), this amount formed was 0.4 molar equivalent (0.0023 mol, 0.64 g).

### Example 3: Measurements of the Interface Tensions (in the Case of the Sunflower Oil-Water System) for Compositions GN12, GN18, AlgN12, Algn18 AND Crude Algn12

The interface properties of the surfactant compositions GN12, GN18, AlgN12, AlgN18 and crude Algn12 were evaluated by measuring the oil-water interface tensions. The surfactants were dissolved in sunflower oil at concentrations ranging from 0.12 to 2.28 g/L. In order to promote the solubility of the surfactants in the oil, the solutions were left in an ultrasonic bath for 10 minutes at 50° C.

The measurements of the interface tensions between the oil and water were taken at 25° C. with a ring tensiometer (Krüss, model K 100 C). The ring used was made of calibrated iridium platinum.

The interface tension between sunflower oil (Carrefour brand) and water at 25° C. ranged between 24.71 and 25.04 mN/m.

For each surfactant composition, the machine initially measured the surface tension of sunflower oil containing the surfactant (low-density liquid) and then the surface tension of water (high-density liquid). Finally, the oil was added delicately to the water, while avoiding the formation of bubbles, and the machine began measuring the interface tension between the sunflower oil and the water (mean of 10 measurements).

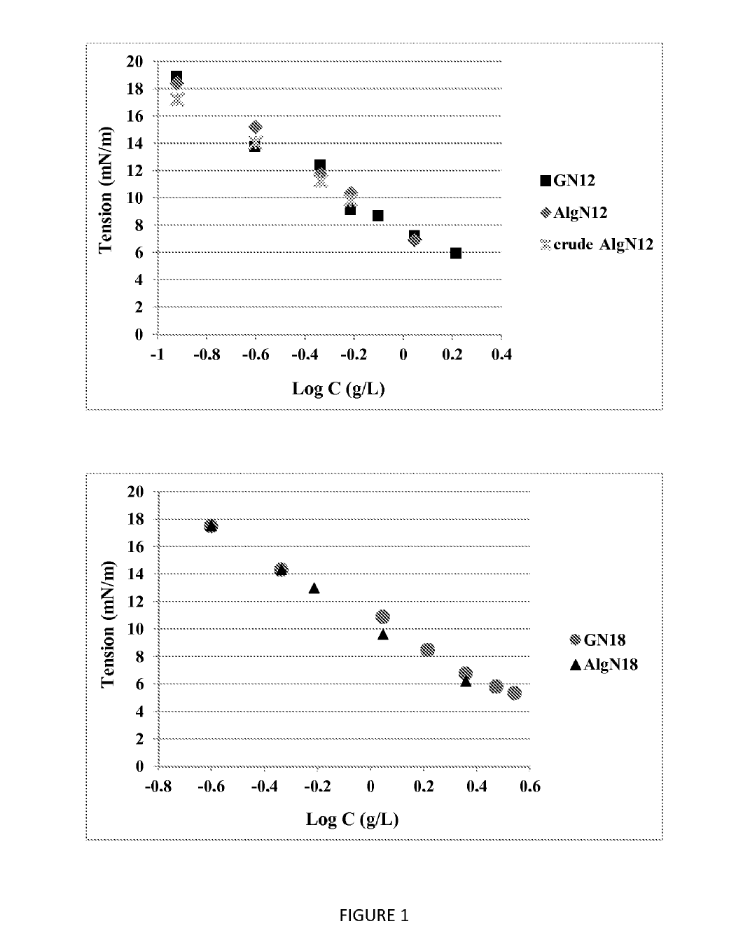

FIG. 1 shows that the surfactant compositions were capable of similarly reducing the water/oil interface tensions to values low enough to give the compositions emulsifying power. It was found that the composition crude AlgN12 obtained via the direct process for obtaining alkyl L-guluronamides and D-mannuronamides from alginate, with prior neutralization of the medium before the aminolysis reaction, was more efficient than the surfactant composition AlgN12 corresponding to the mixture of alkyl L-guluronamide and D-mannuronamide surfactants purified by chromatography (direct process for obtaining alkyl L-guluronamides and D-mannuronamides from alginate, without prior neutralization of the medium before the aminolysis reaction). These results showed the possibility of using a crude reaction product without the need for additional purification. Similarly, the compositions derived from dodecylamine (GN12, AlgN12, crude AlgN12) were more efficient than those derived from octadecylamine (GN18, AlgN18).

### Example 4: Measurement of the Emulsifying Power of Compositions GN12, GN18, AlgN12, Algn18 AND Crude Algn12

The stability of the oil-in-water (O/W) and water-in-oil (W/O) emulsions formed from the surfactant compositions GN12, GN18, AlgN12, AlgN18 and crude Algn12 was studied in comparison with that of commercial alkylpolyglycosides: Montanov 82® from SEPPIC and Xyliance® from Soliance/ARD.

The stability of the two types of O/W and W/O emulsions was evaluated by considering the two water/oil ratios 75/25 and 25/75, respectively, in round-bottomed graduated tubes: 0.5% of the surfactant product is introduced (20 mg). The sunflower oil was introduced (1 or 3 mL) and the surfactants were then dissolved in an ultrasonic bath for 10 minutes at 50° C. After dissolution of the emulsifier, ultrapure water was added (1 or 3 mL).

The two phases were then emulsified using an IKA Ultra-Turrax® T18 basic homogenizer for 10 minutes at 11 000 rpm. The emulsion was placed in a bath thermostatically maintained at 20° C.

The evolution of the emulsion and its gradual demixing was observed for a few hours to several weeks.

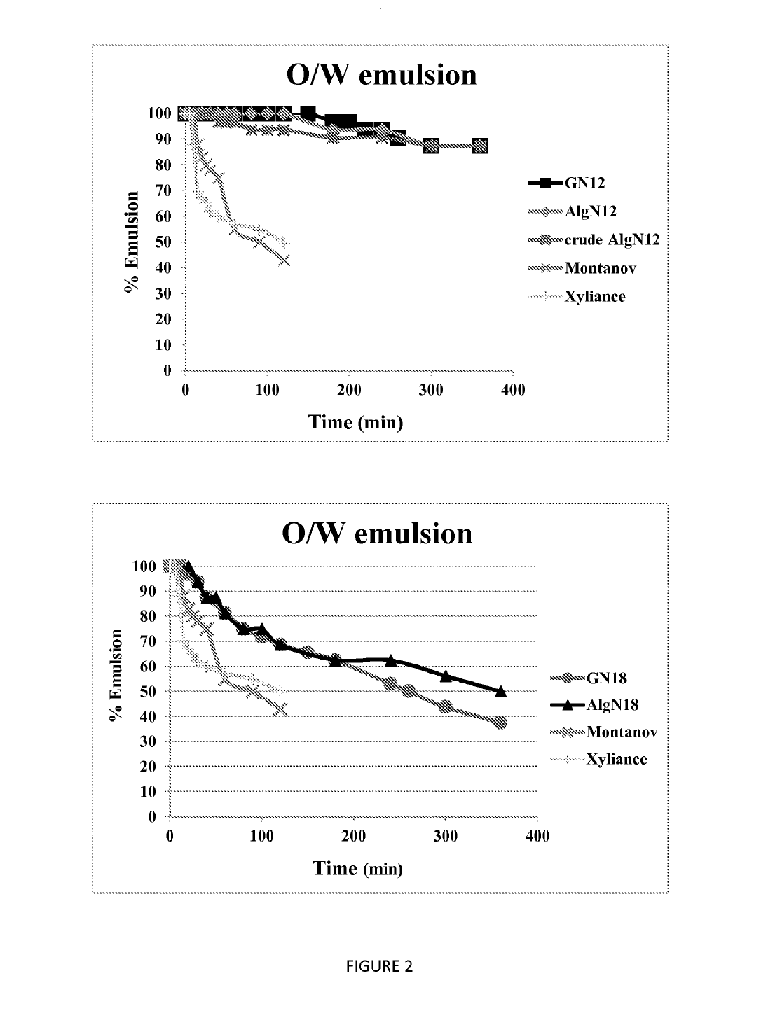

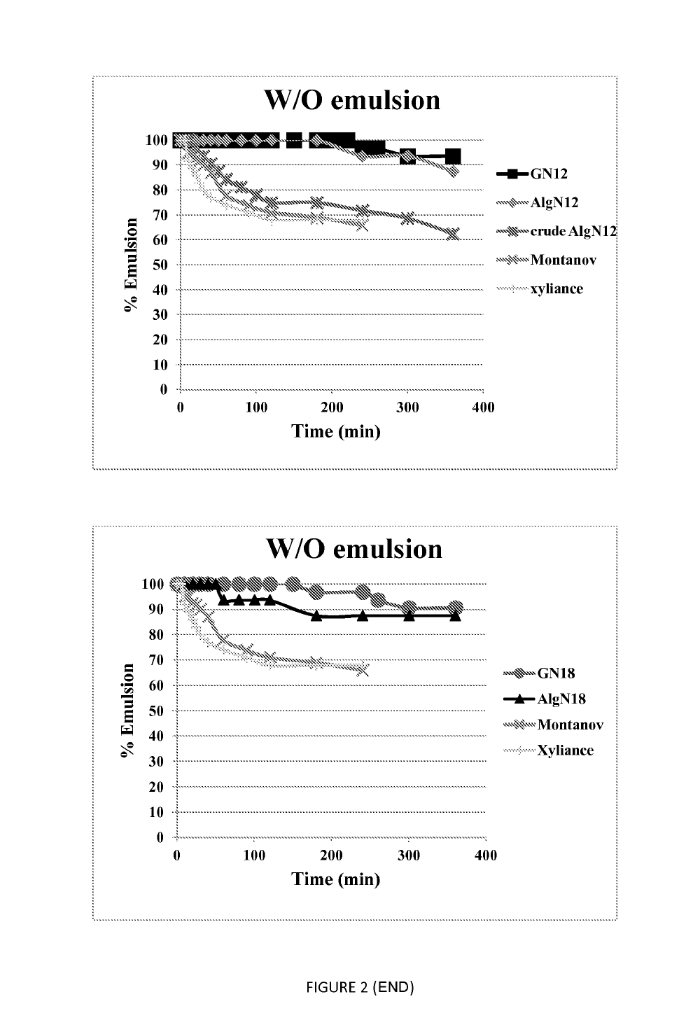

FIG. 2 shows the results of analysis of the emulsifying power of the compositions of some embodiments.

The surfactant compositions derived from dodecylamine (GN12, AlgN12 and crude AlgN12) formed very stable O/W emulsions, including in the case of the crude surfactant composition (crude AlgN12) and also stable W/O emulsions (GN12 and AlgN12). The surfactant compositions derived from octadecylamine (GN18 and AlgN18) formed very stable W/O emulsions; the other O/W emulsions underwent total demixing after 5 hours.

For the two types of emulsions (W/O and O/W), the novel compositions (GN12, AlgN12 and crude AlgN12) formed emulsions that were more stable than the commercial references (Xyliance® and Montanov®).

### Example 5: Antibacterial Activity of Various Fractions of Modified Products

Two protocols were used. The first (protocol A) was applied to fractions enriched in N-(12-dodecyl)-n-butyl α-L-guluronamide isomers by chromatography on silica gel. The second (protocol B) was followed to test the activity of the surfactant compositions derived from dodecylamine (GN12, AlgN12 and crude AlgN12) and of the surfactant composition derived from octadecylamine (AlgN18).

### Protocol A

### 1) Preparation of the Culture Medium:

The culture medium used was a mixture of 21 g/L of Muller-Hinton broth and 10 g/L of agar in water. This mixture was stirred and then left to boil. Next, a step of autoclaving of this mixture, for 30 minutes, was desired in this embodiment so as to sterilize it before any manipulation. This culture medium was poured hot into Petri dishes and then left to cool.

### 2) Preparation of the Modified Test Products:

5 mg of each modified sugar were dissolved in 1 mL of DMSO. Twofold serial dilution with DMSO was then performed using the stock solution, so as to obtain concentrations of 2.5 g/L, 1.25 g/L, 0.625 g/L and 0.3125 g/L.

### 3) Preparation of the Bacterial and Fungal Suspensions:

The bacterial strains used were Pseudomonas aeruginosa, Escherichia Coli, Enterococcus faecium and Staphylococcus aureus, and also the fungal strain Candida albicans. 10⁶ bacteria were taken and then transferred into 0.9% NaCl solution. Each Petri dish, containing Muller-Hinton medium, was flooded with a different bacterial suspension.

### 4) Protocol:

After allowing the bacterial suspensions to dry on the agar, 10 μL of each test solution, and at various concentrations, were deposited on the surface of the agar flooded with the bacterial suspension. 10 μL of DMSO were placed in each Petri dish as a negative control.

The positive controls used were disks soaked with ampicillin for Escherichia coli and Enterococcus faecium, ceftazidime disks for Pseudomonas aeruginosa and vancomycin disks for Staphylococcus aureus.

After drying, the Petri dishes were finally incubated at 37° C. in an oven, for 24 hours. The antibacterial activity was evaluated by measuring the clarification zone in millimeters around the point of deposition of the various test solutions.

By way of example, the activity of fractions I, II and III enriched in isomers of N-(12-dodecyl)-n-butyl α-L-guluronamide (β-L-furanoside form=Fraction I; α-L-pyranoside form=Fraction II; β-L-pyranoside=Fraction III):

| Fractions | | P. | E. | E. | C | S. |
|---|---|---|---|---|---|---|
| enriched in | Concentration | aeruginosa | coli | faecium | albicans | aureus |
| N-(12- | 5 | 0 | 0 | 9 mm | 4.5 mm | 10.5 mm |
| dodecyl)-n- | 2.5 | | | 9 mm | 4 mm | 5 mm |
| butyl-β-L- | 1.25 | | | 7 mm | 4 mm | 5 mm |
| gulurofurano- | 0.625 | | | 0 | 4 mm | 0 |
| siduronamide | 0.3125 | | | 0 | 0 | 0 |
| (75%) | | | | | | |
| Fraction I | | | | | | |
| N-(12- | 5 | 0 | 0 | 20 mm | 5 mm | 18.5 mm |
| dodecyl)-n- | 2.5 | | | 20 mm | 3 mm | 17 mm |
| butyl-α-L- | 1.25 | | | 11 mm | 3 mm | 5 mm |
| guluropyrano- | 0.625 | | | 8 mm | 0 | 0 |
| siduronamide | 0.3125 | | | 8 mm | 0 | 0 |
| (74%) | | | | | | |
| Fraction II | | | | | | |
| N-(12- | 5 | 0 | 0 | 0 | 9 mm | 20.5 mm |
| dodecyl)-n- | 2.5 | | | | 7 mm | 18 mm |
| butyl-β-L- | 1.25 | | | | 5 mm | 0 |
| guluropyrano- | 0.625 | | | | 0 | 0 |
| siduronamide | 0.3125 | | | | 0 | 0 |
| (94%) | | | | | | |
| Fraction III | | | | | | |
| Positive | — | Ceftazidime | Ampicillin | Ampicillin | — | Vancomycin |
| control | | 28 mm | 22 mm | 26 mm | | 19 mm |

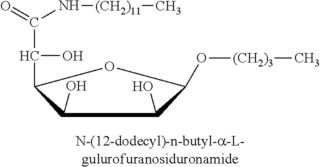

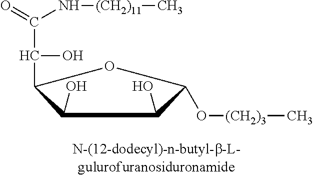

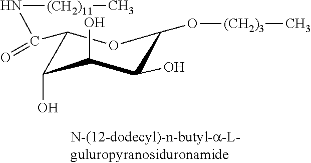

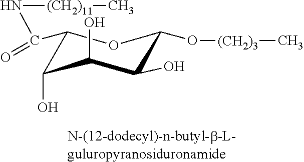

| Fractions | | P. | E. | E. | C | S. |
|---|---|---|---|---|---|---|
| enriched in | Concentration | aeruginosa | coli | faecium | albicans | aureus |
| N-(18- | 5 | 0 | 0 | 6 mm | 0 | 0 |
| octadecyl)- | 2.5 | | | 6 mm | | |
| n-butyl-α-L- | 1.25 | | | 6 mm | | |
| gulurofurano- | 0.625 | | | 4 mm | | |
| siduronamide | 0.3125 | | | 0 | | |
| (47%) | | | | | | |
| N-(18- | 5 | 0 | 0 | 4.5 mm | 0 | 10 mm |
| octadecyl)- | 2.5 | | | 4 mm | | 0 |
| n-butyl-β-L- | 1.25 | | | 4 mm | | 0 |
| gulurofurano- | 0.625 | | | 3 mm | | 0 |
| siduronamide | 0.3125 | | | 0 | | 0 |
| (63%) | | | | | | |
| N-(18- | 5 | 0 | 0 | 6 mm | 0 | 0 |
| octadecyl)- | 2.5 | | | 4 mm | | |
| n-butyl-β-L- | 1.25 | | | 0 | | |
| guluropyrano- | 0.625 | | | 0 | | |
| siduronamide | 0.3125 | | | 0 | | |
| (86%) | | | | | | |
| Positive | — | Ceftazidime | Ampicillin | Ampicillin | — | Vancomycin |
| control | | 28 mm | 22 mm | 26 mm | | 19 mm |

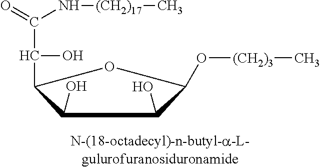

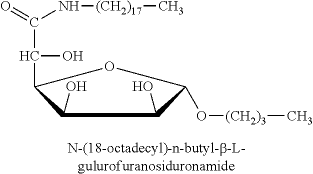

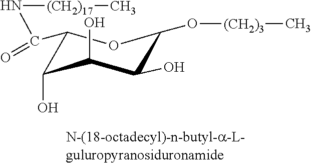

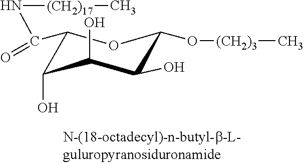

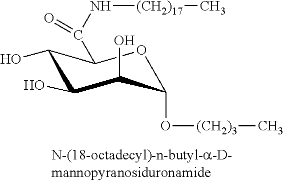

For the studies of the antibacterial and antifungal activities, the fractions derived from the purification of crude N-(12-dodecyl)-n-butyl−L-guluronamide were tested. Each fraction is more enriched in one isomer than the others.

Against the Gram-positive bacteria Enterococcus faecium and Staphylococcus aureus, N-(12 dodecyl)-n-butyl L-guluronamide (fraction enriched in α pyranose form) showed a substantial capacity for inhibiting the growth of these two bacteria, of the order of 20 mm and 18.5 mm, respectively. This inhibitory activity decreased as the concentration decreased. It appears that the pyranose forms, which were predominantly present (94%), had no inhibitory effect against Enterococcus faecium, but showed a greater effect on the growth of the yeast Candida albicans (9 mm at 5 g/L), in addition to the strongest inhibitory effect against Staphylococcus aureus (20.5 mm at 5 g/L).

On the other hand, N-(18 octadecyl)-n-butyl L-guluronamide showed no inhibitory effect either on the Gram-positive bacterium Staphylococcus aureus or on the yeast Candida albicans.

Against the Gram-negative bacteria Pseudomonas aeruginosa and Escherichia coli, neither the N-(12 dodecyl)-n-butyl L-guluronamide nor the N-(18 octadecyl)-n-butyl L-guluronamide (whether in furanose or pyranose form) showed any inhibitory activity on the growth of these two bacteria.

Protocol B

The advantageous results of the antibacterial and antifungal activity, obtained for the fractions enriched in N-(12-dodecyl)-n-butyl α-L-guluronamide isomers, encouraged the applicants to test the antibacterial and antifungal activity of mixtures of surfactant products obtained from guluronates (GN12) and from alginates (AlgN12, crude AlgN12 and AlgN18). For these surfactant mixture products, a new method was tested based on the study of the capacity of the surfactants of some embodiments to kill the bacteria, followed by counting of the number of live bacteria on Muller-Hinton agar.

1) Preparation of the Bacterial and Fungal Inoculum:

The inoculum was prepared at a turbidity equivalent to 0.5 MacFarland (Biomerieux France), and then diluted to 1/100 (10⁶ CFU/ml). From this inoculum, a series of dilutions to 10⁻¹, 10⁻², 10⁻³, 10⁻⁴, 10⁻⁶ and 10⁻⁶ was prepared. 100 μl of each dilution were spread (counting method) onto the surface of a Muller-Hinton agar (determination of the number of bacteria in CFU/ml in the inoculum ‘N’.

2) Preparation of the Modified Test Products:

Stock solutions were prepared for each surfactant GN12 (430 mg/ml), AlgN12 (310 mg/ml), crude AlgN12 (216 mg/ml) and AlgN18 (219 mg/ml). Twofold serial dilution with DMSO was performed for each product in Muller-Hinton broth: the final dilution was 1/128.

3) Protocol:

1 ml of the bacterial inoculum was added to each tube of the surfactant dilutions.

After incubation for 24 hours at 36° C., 100 μl of each clear tube were spread onto the surface of a Muller-Hinton agar, followed by incubation for 24 hours at 37° C.

The number of live bacteria was determined: N0=n×10 CFU/ml (n=number of colonies).

The percentage of live bacteria was calculated: N0/N×100:

| | Name of | Concentration | |
|---|---|---|---|
| Name of the | the | prepared | |
| bacterium | product | (mg/ml) | Results |
| Escherichia | AlgN₁₂ | 310 mg/ml | 155 mg/ml => Inhibition of 100% of the bacteria |
| coli | | | 77.5 mg/ml => Inhibition of 100% of the bacteria |
| | | | 38.75 mg/ml => Inhibition of 100% of the bacteria |
| | | | 19.375 mg/ml => Inhibition of 99.99% of the |
| | | | bacteria |
| | AlgN₁₂ | 216 mg/ml | 108 mg/ml => Inhibition of 100% of the bacteria |
| | (Crude) | | 54 mg/ml => Inhibition of 100% of the bacteria |
| | | | 27 mg/ml => Inhibition of 100% of the bacteria |
| | | | 13.5 mg/ml => Inhibition of 99.99% of the bacteria |
| | AlgN₁₈ | 219 mg/ml | 109.5 mg/ml => Inhibition of 100% of the bacteria |
| | | | 54.75 mg/ml => Inhibition of 100% of the bacteria |
| | | | 27.375 mg/ml => Inhibition of 99.99% of the |
| | | | bacteria |
| | GN₁₂ | 430 mg/ml | 215 mg/ml => Inhibition of 100% of the bacteria |
| | | | 107.5 mg/ml => Inhibition of 99.99% of the bacteria |
| Pseudomonas | AlgN₁₂ | 310 mg/ml | 155 mg/ml => Inhibition of 100% of the bacteria |
| aeruginosa | | | 77.5 mg/ml => Inhibition of 100% of the bacteria |
| | | | 38.75 mg/ml => Inhibition of 100% of the bacteria |
| | | | 19.375 mg/ml => Inhibition of 99.9% of the bacteria |
| | AlgN₁₂ | 216 mg/ml | 108 mg/ml => Inhibition of 100% of the bacteria |
| | (Crude) | | 54 mg/ml => Inhibition of 100% of the bacteria |
| | | | 27 mg/ml => Inhibition of 100% of the bacteria |
| | | | 13.5 mg/ml => Inhibition of 99.9% of the bacteria |
| | AlgN₁₈ | 219 mg/ml | 109.5 mg/ml => Inhibition of 100% of the bacteria |
| | | | 54.75 mg/ml => Inhibition of 100% of the bacteria |
| | | | 27.375 mg/ml => Inhibition of 100% of the bacteria |
| | GN₁₂ | 430 mg/ml | 215 mg/ml => Inhibition of 100% of the bacteria |
| | | | 107.5 mg/ml => Inhibition of 100% of the bacteria |
| | | | 53.75 mg/ml => Inhibition of 100% of the bacteria |
| Enterococcus | AlgN₁₂ | 310 mg/ml | 2.42 mg/ml => Inhibition of 100% of the bacteria |
| faecium | AlgN₁₂ | 216 mg/ml | 1.7 mg/ml => Inhibition of 100% of the bacteria |
| | (Crude) | | |
| | AlgN₁₈ | 219 mg/ml | 109.5 mg/ml => Inhibition of 100% of the bacteria |
| | | | 54.75 mg/ml => Inhibition of 100% of the bacteria |
| | | | 27.375 mg/ml => Inhibition of 100% of the bacteria |
| | GN₁₂ | 430 mg/ml | 3.36 mg/ml => Inhibition of 100% of the bacteria |
| Candida | AlgN₁₂ | 310 mg/ml | 2.42 mg/ml => Inhibition of 100% of the bacteria |
| albicans | AlgN₁₂ | 216 mg/ml | 1.7 mg/ml => Inhibition of 100% of the bacteria |
| | (Crude) | | |
| | AlgN₁₈ | 219 mg/ml | 109.5 mg/ml => Inhibition of 100% of the bacteria |
| | | | 54.75 mg/ml => Inhibition of 100% of the bacteria |
| | | | 27.375 mg/ml => Inhibition of 100% of the bacteria |
| | GN₁₂ | 430 mg/ml | 3.36 mg/ml => Inhibition of 100% of the bacteria |
| Staphylococcus | AlgN₁₈ | 219 mg/ml | 109.5 mg/ml => Inhibition of 100% of the bacteria |
| aureus | | | |

Against the Gram-positive bacterium Enterococcus faecium, the mixture of crude AlgN12 (with neutralization prior to the aminolysis reaction) appears to be more efficient than the mixture AlgN12 (process without neutralization prior to the aminolysis reaction) since a concentration of 1.7 mg/mL of AlgN12 (crude) is sufficient to inhibit 100% of Enterococcus faecium, whereas this concentration is higher in the case of AlgN12 (2.42 mg/ml). Furthermore, the mixture of surfactants derived from the chemical modification of alginate (mixture of mannuronamide and guluronamide) has stronger power against Enterococcus faecium than the surfactants based on guluronamides alone (higher concentration of GN12 of the order of 3.36 mg/mL to inhibit 100% of Enterococcus faecium). This observation is the same for the inhibition of Candida albicans.

Against the Gram-negative bacteria Pseudomonas aeruginosa and Escherichia coli, very high concentrations of surfactants may be required to inhibit these two bacteria to 100%; showing that AlgN12 (38.75 mg/ml), crude AlgN12 (13.5 mg/ml) and GN12 (53.75 mg/ml) had low antibacterial power against these two types of bacteria.

By comparing AlgN12 and AlgN18, the alginate amide surfactant product bearing an octadecyl chain has lower antibacterial power against the Gram-positive and Gram-negative bacteria tested. A high concentration of AlgN18 may be necessary to inhibit 100% of the bacteria Enterococcus faecium (27.375 mg/ml), Pseudomonas aeruginosa (27.375 mg/ml), Escherichia coli (54.75 mg/ml) and Candida albicans (27.375 mg/ml).

### LIST OF REFERENCES

1—Hill and Lehen-Ferrenbach, “In Sugar-based surfactants fundamentals and Applications”, C. C. Ruiz (Ed.), 1-20, CRC Press, ISBN 978-1-4200-5166-7, 2009
2—Laurent et al., J. Surfact. Deterg., 14: 51-63, 2011
3—International patent application WO 92/06984
4—International patent application WO 93/03004
5—U.S. Pat. No. 7,655,611
6—U.S. Pat. No. 5,872,111
7—U.S. Pat. No. 2,670,345
8—Benvegnu and Sassi, Topics in Current Chemistry, 294: 143-164, 2010
9—International patent application WO 03/104 248
10—International patent application WO 03/099 870
11—International patent application WO 2009/134 368
12—International patent application WO 98/40511

1. A process for preparing a composition, the process comprising:
(i) alkyl L-guluronamides of formulae (Ia) and (Ib): 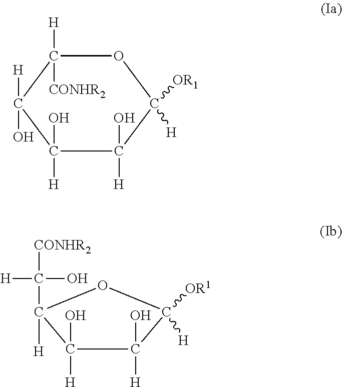
or
(ii) a mixture of alkyl L-guluronamides of formulae (Ia) and (Ib) and of alkyl D-mannuronamides of formulae (IIa) and (IIb): 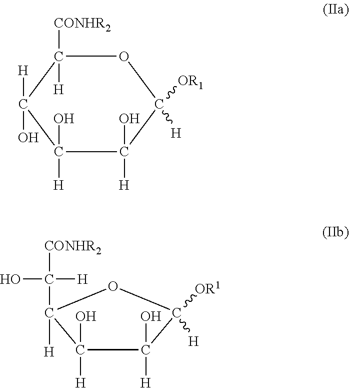
wherein:
R₁ is a linear or branched, saturated or unsaturated alkyl chain of 2 to 22 carbon atoms; and
R₂ is a hydrogen atom, a linear or branched, saturated or unsaturated alkyl chain of 2 to 22 carbon atoms which may comprise a terminal amine function; wherein the process further includes:
a) performing a butanolysis and Fischer glycosylation reaction using poly(oligo)guluronates, oligoalginates, alginates and/or brown algae; and
b) performing a aminolysis reaction on the reaction medium derived from the butanolysis and Fischer glycosylation reaction, in the presence of a linear or branched, saturated or unsaturated amine of formula R′NH₂ wherein R′ is composed of 2 to 22 carbon atoms.

2. The process as claimed in claim 1, further including performing of neutralizing the reaction medium derived from the butanolysis and Fischer glycosylation reaction before the aminolysis reaction.

3. The process as claimed in claim 1, wherein the butanolysis and Fischer glycosylation reaction is performed in the presence of:
(i) water and/or of an ionic solvent and/or of a eutectic solvent,
(ii) a linear or branched, saturated or unsaturated alcohol of the formula ROH, containing from 1 to 4 carbon atoms, and
(iii) an acid catalyst.

4. The process as claimed in claim 3, wherein the acid catalyst is selected from the group consisting of consisting of: hydrochloric acid, sulfuric acid, an alkylsulfuric acid, a sulfonic acid, an alkylsulfonic acid or an alkyl sulfosuccinate, perhalohydric acids, metals, oxides thereof or salts thereof such as the halides thereof.

5. The process as claimed in claim 4, wherein the acid catalyst is methanesulfonic acid.

6. The process as claimed in claim 3, wherein the alcohol ROH is n-butanol.

7. The process as claimed in claim 1, wherein the aminolysis reaction is performed in the presence of a fatty amine selected from the group consisting of dodecylamine and oleylamine.

8. A composition obtained via the process as claimed in claim 1.

9. The composition as claimed in claim 8, wherein the composition is an oil-in-water or water-in-oil emulsion.

10. The use of the composition as claimed in claim 8 as a surfactant.

11. The use of a composition as claimed in claim 10, wherein the surfactant is selected from the group consisting of solubilizers, hydrotropes, wetting agents, foaming agents, emulsifying agents, emulsifiers and/or detergents.

12. The use of a composition as claimed in claim 8 as an antibacterial and/or antifungal agent.

13. A surfactant, comprising:
the composition as claimed in claim 8.

14. An antibacterial and/or antifungal, comprising:
the composition as claimed in claim 8.

15. The process as claimed in claim 2, wherein the butanolysis and Fischer glycosylation reaction is performed in the presence of:
(i) water and/or of an ionic solvent and/or of a eutectic solvent,
(ii) a linear or branched, saturated or unsaturated alcohol of formula ROH, containing from 1 to 4 carbon atoms, and
(iii) an acid catalyst.

16. The process as claimed in claim 15, wherein the acid catalyst is selected from the group consisting of: hydrochloric acid, sulfuric acid, an alkylsulfuric acid, a sulfonic acid, an alkylsulfonic acid or an alkyl sulfosuccinate, perhalohydric acids, metals, oxides thereof or salts thereof such as the halides thereof.

17. The process as claimed in claim 16, wherein the acid catalyst is methanesulfonic acid.

18. The process as claimed in claim 15, wherein the alcohol ROH is n-butanol.

19. The process as claimed in claim 2, wherein the aminolysis reaction is performed in the presence of a fatty amine selected from the group consisting of dodecylamine and oleylamine.

20. The process as claimed in claim 1, wherein the aminolysis reaction is performed in the presence of a fatty amine selected from the group consisting of dodecylamine and oleylamine.
